(19) United States
(12) Patent Application Publication
(10) Pub. No.: US 2021/0178130 A1
(43) Pub. Date: Jun. 17, 2021

(54) ASYMMETRICAL BALLOON CATHETER

(71) Applicant: Embark Medical, LLC, Santa Clara, CA (US)

(72) Inventor: Ray Betelia, San Jose, CA (US)

(21) Appl. No.: 16/752,350

(22) Filed: Jan. 24, 2020

Related U.S. Application Data
(60) Provisional application No. 62/949,348, filed on Dec. 17, 2019.

(51) Int. Cl. A61M 25/10

(52) U.S. Cl. CPC A61M 25/1002; A61M 2025/1079; A61M 2025/1061; A61M 25/1025

(57) ABSTRACT

An example apparatus embodiment includes a catheter body and an inflatable balloon having longitudinal edges sealed to the catheter body. The balloon may be configured to extend farther outward from an outer surface of the catheter body in a first direction when inflated than a second direction opposite the first direction. The catheter body may be formed with a lumen for controlling the balloon inflation, another lumen for transporting fluid to and from a diagnostic or therapeutic site in a patient's body, and yet another lumen for transporting a therapeutic or diagnostic instrument or other payload.

### FIELD OF THE INVENTION

Embodiments are related in general to medical catheters and more specifically to catheters with an inflatable balloon.

### BACKGROUND

A balloon catheter for medical procedures may include an inflatable balloon attached to a catheter body. Fluid may be forced into the balloon through a lumen in the catheter to inflate the balloon. Fluid may be removed from the balloon through the lumen to deflate the balloon. While the balloon is being inflated or deflated, the lumen in the catheter may not be available for other uses. Using the same lumen for adjusting balloon inflation, transporting therapeutic or diagnostic devices or materials, and delivering or removing fluids may substantially increase the amount of time needed to complete a medical procedure. The increase in time may be associated with undesirable medical complications. For example, imaging contrast media may be delivered through the lumen of the catheter. Imaging contrast media be toxic to kidneys and other body organs. A delay in removing the imaging contrast media following a medical procedure, or failure to remove all of the contrast media, may allow some of the contrast media to migrate away from the imaging site.

Balloon catheters previously known in the art may have a balloon which extends outward in all directions from an approximately cylindrical catheter body. Such balloons entirely surround the outer circumference of the catheter body. A balloon extending outward in all directions from the outer surface of the catheter body may be difficult to position in some body lumens or may apply pressure to the walls of a body lumen where pressure may be unneeded or ineffective for treatment or diagnostic purposes.

### BRIEF DESCRIPTION OF THE DRAWINGS

FIG. 1 a side view of an example catheter embodiment having an inflated balloon extending asymmetrically outward from an outer surface of the catheter.

FIG. 2 is a partial enlarged view of the distal end of the example balloon catheter of FIG. 1.

FIG. 3 is a view toward the distal end of the example balloon catheter of FIGS. 1-2.

FIG. 4 is a side view of the example balloon catheter of the previous figures, showing the balloon in an example of a deflated and stowed position.

FIG. 5 is a cross-sectional view A-A showing examples of some internal features of the balloon catheter, including an example of a balloon inflation fluid in the interior volume of the example of an inflated balloon and an example of a fluid to be transported through a fluid transport lumen. A location and viewing direction for the cross-section is marked by a section line A-A in FIG. 2.

FIG. 6 is a cross-sectional view B-B showing an example of a balloon in a deflated and/or stowed position against the outer surface of the catheter body. The balloon fill lumen, balloon fill port, fluid transport lumen, and external fluid ports are shown empty in the example of FIG. 6, without the examples of fluids shown in FIG. 5. A location and viewing direction for the cross-sectional view is marked by a section line B-B in FIG. 4.

FIG. 7 is an alternative cross-sectional view B-B showing another example of the balloon in a deflated and/or stowed position against the outer surface of the catheter body.

FIG. 8 is another alternative cross-sectional view B-B showing another example of a deflated and/or stowed position of the balloon against the outer surface of the catheter body.

FIG. 9 is an alternative cross-sectional view A-A showing another example of an inflated balloon extending asymmetrically outward from the catheter body.

FIG. 10 is a partial enlarged view of the distal end of the example balloon catheter of FIG. 1, showing an example of perfusion and/or aspiration ports having different sizes.

FIG. 11 is a partial enlarged view of the distal end of the example balloon catheter of FIG. 1, showing an example of perfusion and/or aspiration ports having an arcuate perimeter shape.

FIG. 12 is a partial enlarged view of the distal end of the example balloon catheter of FIG. 1, showing an example of perfusion and/or aspiration ports having an arcuate perimeter shape that differs from the arcuate shape example in FIG. 11.

FIG. 13 is a partial enlarged view of the distal end of the example balloon catheter of FIG. 1, showing an example of perfusion and/or aspiration ports arranged in port groups.

FIG. 14 is a partial enlarged view of the distal end of the example balloon catheter of FIG. 1, showing an example of perfusion and/or aspiration ports configured as narrow slots or slits.

FIG. 15 is a partial enlarged view of the distal end of an example balloon catheter having a tapered distal end, and further illustrating an example of the balloon in a deflated and/or stowed position.

FIG. 16 is a cross-sectional view C-C showing examples of some internal features of the example catheter of FIG. 1. A location and viewing direction for cross-sectional view C-C is marked by a section line C-C in FIG. 3.

### SUMMARY

An example apparatus embodiment of a balloon catheter includes a catheter body and an inflatable balloon having longitudinal edges sealed to the catheter body. The balloon is configured to extend laterally outward from an outer surface of the catheter body farther in a first direction when inflated than a second direction. In some example embodiments of the balloon catheter, the first direction may be radially outward from the outer surface of the catheter body and the second direction may be radially opposite the first direction.

An example balloon catheter embodiment may include a catheter body formed with a balloon fill lumen in fluid communication with an interior volume of the balloon and a working lumen extending through a proximal end of the catheter body to a distal end of the catheter body. The catheter body may be formed with a balloon fill port in fluid communication with the balloon fill lumen and the interior volume of the balloon. In some example embodiments of the balloon catheter, the balloon fill lumen is not in fluid communication with the working lumen.

An example catheter body may further be formed with a fluid transport lumen and a plurality of external fluid ports passing through the outer surface of the catheter body into the fluid transport lumen. In some example embodiments, the catheter body may be formed with some or all of the external fluid ports adjacent a first longitudinal edge of the balloon. An example balloon catheter embodiment may optionally be formed with some or all of the external fluid ports positioned on the outer surface of the catheter body opposite the balloon. In some example embodiments of the balloon catheter, the fluid transport lumen is not in fluid communication with the working lumen. In some example embodiments of the balloon catheter, the balloon fill lumen is not in fluid communication with the fluid transport lumen.

An example balloon catheter embodiment may optionally include a luer assembly attached to the catheter body. The luer assembly may include a first hub in fluid communication with the balloon fill lumen; a second hub in fluid communication with the working lumen; and a third hub in fluid communication with the fluid transport lumen. Any one or more of the first, second, and third hubs may be attached to the catheter body separately from the luer assembly.

The balloon included in an example balloon catheter embodiment may be formed with a tapered distal end and a tapered proximal end. In a preferred example embodiment of a balloon catheter, the balloon when inflated does not cover the external fluid ports. Some example embodiments of the balloon have a balloon wall shaped as a hollow longitudinal cylindrical segment when the balloon is inflated, with the longitudinal edges of the balloon wall sealed to the outer surface of the catheter. A balloon may alternatively be formed with an outer surface that is a segment of a hollow sphere, or with other surface shapes.

An example balloon catheter embodiment may optionally include a radiopaque marker positioned on the catheter body between the balloon and the distal end of the catheter body. An example balloon catheter embodiment may optionally include a radiopaque marker positioned on catheter body adjacent a proximal end of the balloon. A radiopaque marker may optionally be integrally formed with the catheter body or may be attached to a surface of the catheter body.

For some example balloon catheter embodiments, the balloon when inflated covers less than about eighty percent of a circumference of the catheter body. When inflated, the balloon may cover at least twenty percent of said circumference of the catheter body. For an example embodiment of a balloon catheter, the catheter body may be formed with a balloon fill lumen in fluid communication with an interior volume of the balloon, a fluid transport lumen, and more than one of an external fluid port passing through the outer surface along the at least twenty percent of the circumference into the fluid transport lumen.

In some example embodiments of a balloon catheter, the catheter body may be formed with a first external fluid port having a first diameter and a second external fluid port having a second diameter less than the first diameter. Some example embodiments of a catheter body may alternatively have an external fluid port shaped as a narrow slit. The slit may be sufficiently narrow to remain closed unless forced open by sufficient pressure of a fluid in the catheter, for example pressure of a fluid in the fluid transport lumen of the catheter.

Some example embodiments of a balloon catheter are formed with a balloon fill lumen, a fluid transport lumen, a working lumen, and a plurality of external fluid ports passing through a portion of an outer surface of the catheter not covered by the balloon when inflated. The example balloon catheter may be configured for simultaneous transport of a first fluid through the balloon fill lumen, a medical instrument through the working lumen, and a second fluid through the fluid transport lumen.

### DESCRIPTION

Example embodiments of an apparatus include a balloon catheter configured for insertion into body lumens such as arteries, vessels, or lumens in other organs. The disclosed examples of a balloon catheter may further be configured for fluid injection and optionally for fluid evacuation distal to the balloon, proximal to the balloon, and/or at the balloon dilatation region in a body lumen. Balloon catheter embodiments may further be configured to deliver prostheses, pharmaceutical compounds, diagnostic instruments, and surgical instruments through a lumen in the catheter to a site in a body lumen where a therapeutic and/or diagnostic procedure is being performed. A balloon catheter embodiment may optionally carry a pharmaceutical compound, a diagnostic agent or instrument, a therapeutic agent, or a prosthesis on an outer surface of the balloon.

The disclosed examples of a balloon catheter include an inflatable balloon made from an elastic material sealed to an outer surface of a catheter body. In some example embodiments a segment of the inflated balloon forms a hollow elongate cylinder. The balloon may optionally be formed with tapered proximal and distal ends attached to, or alternatively formed as an integral part of, the hollow cylindrical segment. The balloon may be stowed in an uninflated state and folded against the outer surface of the catheter body. The balloon may be held securely against the catheter body by evacuating fluid and gas from the interior volume of the balloon, permitting the catheter and balloon to be advanced smoothly through a body lumen to a selected location, where the balloon may be inflated to perform a medical procedure. After the medical procedure is complete, the balloon may be deflated and held against the catheter body by evacuating the interior of the balloon. The balloon or other parts of the catheter may optionally include features such as micro probes to enhance penetration into a wall of a body lumen.

When inflated, the balloon extends asymmetrically away from the outer surface of the catheter. The asymmetry of the inflated balloon is established by attaching the balloon to the catheter in such a way that the balloon does not entirely surround the outer circumference of the cylindrical catheter body, i.e., a substantial portion of the outer surface of the catheter body in a gap between the attached longitudinal edges of the balloon remains exposed after inflation. The asymmetric balloon and catheter are not concentric with one another, that is, the cylindrical segment of the balloon and the cylindrical catheter do not share a common longitudinal center. The offset attachment position of the asymmetric balloon to the catheter causes the inflated balloon to extend a greater distance outward from the catheter in one direction than in an opposite direction. The shape and asymmetric position of the inflated balloon on the catheter body prevent the inflated balloon from covering the external fluid ports formed along the longitudinal edges of the balloon and reducing fluid flow through the external fluid ports.

A balloon catheter embodiment is preferably formed with separate lumens for inflating the balloon, transporting fluid to a site in the body where a medical procedure is being performed, and optionally for delivering instruments to the site in the body. Changing the balloon inflation, inserting and removing instruments through the catheter, and injecting or evacuating fluid through the catheter may be performed simultaneously or in any preferred order or sequence without removing the catheter from a body lumen. Fluid may flow into and out of the balloon inflation lumen and the fluid transport lumen, and payloads such as instruments, guidewires, and solid, liquid, and/or gaseous materials may be moved in and out of the working lumen.

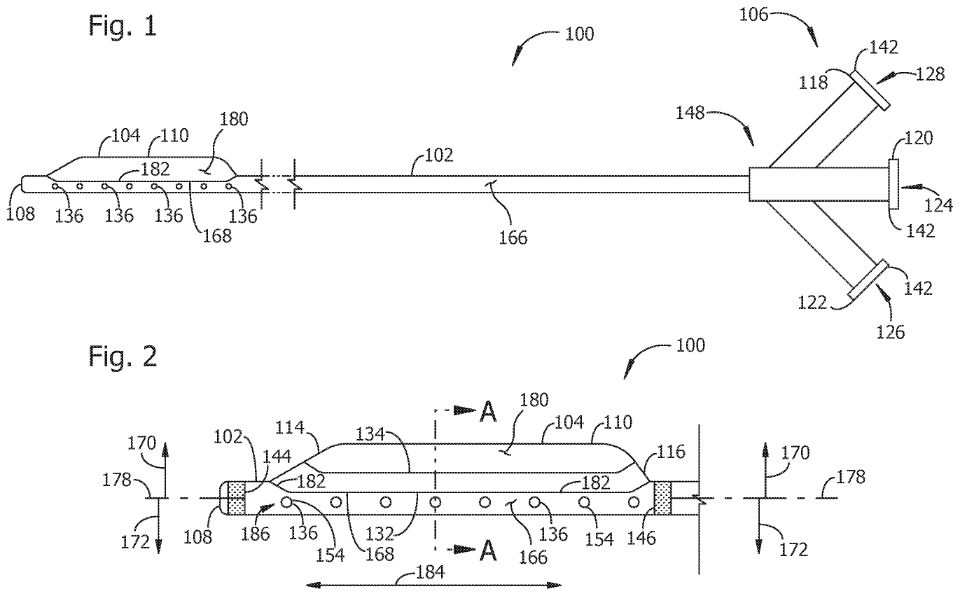

An example balloon catheter embodiment 100 is shown in FIG. 1. A balloon catheter 100 in accord with an apparatus embodiment may be referred to herein as an asymmetrical balloon catheter 100. An example balloon 104 extends asymmetrically outward from a catheter body 102 near a distal end 108. The balloon 104 in FIG. 1 is an example of an inflated balloon 110. At least one and in many embodiments more than one perfusion and/or aspiration ports 136 pass through the longitudinal outer surface 166 of the catheter body 102 adjacent the longitudinal edge 168 of the balloon 104. The perfusion and/or aspiration ports 136 may also be referred to herein as external fluid ports 136. The external fluid ports 136 are separated from one another in a longitudinal direction 184. The external fluid ports 136 may be used, for example, to distribute imaging contrast fluid to a location in a body lumen where a procedure is to be performed and to remove the contrast fluid after imaging is complete. Fluids other than imaging contrast fluid may optionally be delivered and/or removed through the external fluid ports 136.

Unlike catheters having a balloon projecting outward in all directions from the catheter surface and catheters having a balloon surrounding essentially all of the circumference of the catheter when the balloon is inflated, the example catheter embodiments 100 disclosed herein have external fluid ports 136 adjacent the longitudinal edges of the inflated balloon. The external fluid ports 136 in a balloon catheter embodiment 100 may be used to deliver and recover fluids such as imaging contrast fluid while the balloon is fully inflated and while the balloon is being inflated or deflated. Balloon catheter embodiments 100 preferably include at least one longitudinal row 186 of external fluid ports 136 passing through the exposed fraction 174 of the circumference of the outer surface 166 of the catheter body 102 between laterally separated longitudinal edges 168 of the wall 130 of the balloon 104. Balloon catheter embodiments 100 enable fluids to be placed more precisely, in smaller quantities, and recovered more completely and quickly compared to balloon catheters previously known in the art because of the positioning of the external fluid ports 136 along the edges 182 of the balloon wall 130 and the separate lumens for transporting fluid and inflating the balloon.

The asymmetrical extension of the inflated balloon 110 in a first direction 170 more than other directions 172 from the catheter body 102 enables a net contact force per unit area between the outer surface 180 of the balloon and a treatment area in a body lumen to be maximized in the direction of extension 170, possibly with lower magnitudes of contact force in other directions. The net force is the vector sum of contact forces in all directions between the balloon and a surface in a body lumen or tissue or other objects in a body lumen. Furthermore, compared to previously known balloon catheters having a balloon that surrounds the circumference of the catheter body, the example asymmetric balloon catheter embodiments 100 may be easier to maneuver into small body lumens and into a preferred branch of a branching body lumen, for example by selectively inflating and deflating the balloon to deflect the distal end of the catheter in a preferred direction or by pushing the inflated balloon against a surface to deflect the catheter away from the surface.

An example of a luer assembly 148 may be attached to the catheter body 102 near a proximal end 106. The luer assembly includes one or more hubs in fluid communication with corresponding lumens in the catheter body. A first hub 118 is in fluid communication with the interior of the balloon 104 through an intervening balloon fill lumen in the catheter body 102. The first hub 118 may be used to deliver balloon inflation fluid to the interior volume of the balloon to inflate the balloon 104, remove balloon inflation fluid to deflate the balloon, and hold the deflated balloon 104 firmly against the catheter body by evacuating the interior of the balloon. A second hub 120 in fluid communication with the working lumen 124 in the catheter body 102 may be used to deliver and recover prostheses, diagnostic instruments, fluids, and/or surgical instruments to and from a body lumen. A third hub 122 is in fluid communication with the external fluid ports 136 through an intervening fluid transport lumen in the catheter body 102. Each of the examples of the first hub 118, second hub 120, and third hub 122 may further include a luer fitting 142. One or more of the hubs (118, 120, 122) may alternatively be attached to the catheter body separately from the luer assembly 148.

FIG. 2 shows some additional features of the example balloon catheter embodiment of FIG. 1. The example of an inflated balloon 110 may be configured to form a cylindrical outer surface 180 extending away from the outer surface 166 of the catheter body 102 in a first radial direction 170 relative to the longitudinal center axis 178 of the catheter body. The inflated balloon 110 preferably does not extend outward in a second radial direction 172 opposite the first radial direction 170, i.e., the direction of extension of the inflated balloon is asymmetric with respect to the longitudinal center axis 178 of the catheter body 102. The balloon wall 130 forming the outer surface 180 of the inflated balloon 110 attaches to the outer surface 166 of the catheter body 102 at laterally opposing longitudinal edges 168 of the balloon wall 130, with sufficient space provided between the sealed joint 132 along the opposing longitudinal edges 168 for at least one longitudinal row 186 of external fluid ports 136 to penetrate the exposed fraction 174 of the outer surface of the catheter. A balloon catheter embodiment 100 may include one or more of a first longitudinal row 186 of external fluid ports 136 adjacent a first longitudinal edge 168 of the balloon wall 130, an optional second longitudinal row 186 of external fluid ports 136 along a second longitudinal edge 168 laterally opposite the first longitudinal edge 182, and an optional third longitudinal row 186 through the catheter outer surface 166 opposite the inflated balloon 110.

Continuing with the example of FIG. 2, a distal radiopaque marker 144 may be positioned between the tapered distal end 114 of the balloon 104 and the distal end 108 of the catheter body 102. A proximal radiopaque marker 146 may be positioned proximally to the tapered proximal end 116 of the balloon 104 and proximally to the external fluid ports. The radiopaque markers may be formed as separate parts and attached to the catheter by welding or adhesive, may be electroplated or deposited from a liquid solution onto the catheter body, or may be integrally molded into the body of the catheter. A tungsten band and a tungsten-filled polymer material are examples of radiopaque markers, although other radiopaque materials may be used. All of the external fluid ports 136 may optionally be positioned between the distal radiopaque marker 144 and the proximal radiopaque marker 146.

The balloon 104 is strongly secured to the outer surface 166 of the catheter body 102 at a sealed joint 132 formed by attaching the material of the balloon wall to the material of the catheter body. The attachment between the balloon wall 130 and the outer surface 166 of the catheter preferably forms an uninterrupted liquid-tight and gas-tight bond all the way around the contact perimeter between the balloon and catheter. The sealed joint 132 may be formed by thermal welding, solvent welding, ultrasonic welding, laser welding, and/or adhesive bonding.

As suggested in FIG. 2, the balloon 104 may optionally be formed with a fold line or crease 134 to encourage the balloon to fold smoothly against the surface of the catheter when the balloon is deflated to its stowed position. The example crease 134 may be formed by subjecting the folded balloon to heat and pressure against the surface of a mandrel or by molding a ridge or groove into the balloon wall.

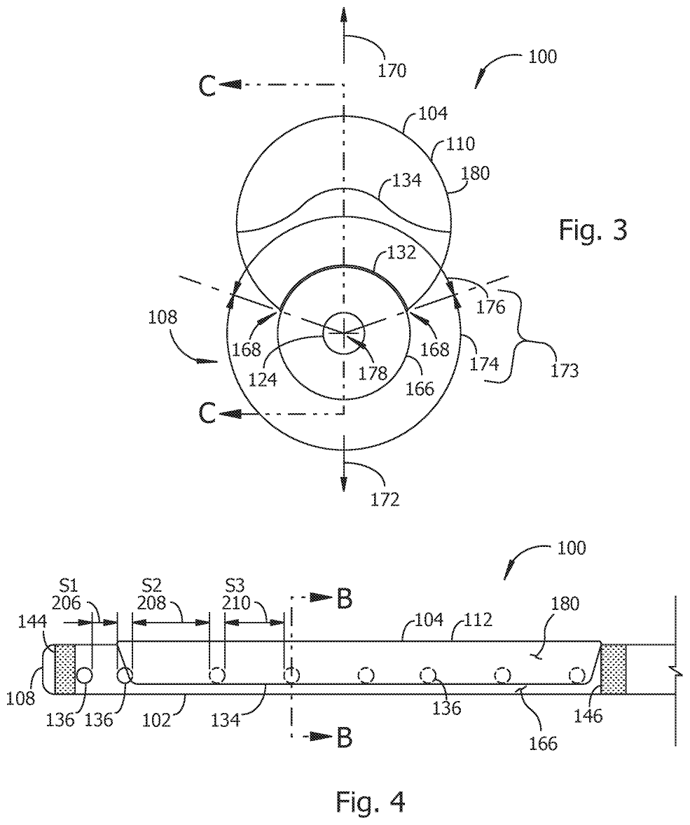

FIG. 3 shows a view toward the distal end of an example asymmetric balloon catheter embodiment 100. The inflated balloon 110 extends asymmetrically away from the outer surface 166 and longitudinal center axis 178 of the catheter body 102. An instrument lumen 124, also referred to herein as a working lumen 124, passes through the distal end 108 of the catheter.

FIG. 3 further illustrates the asymmetry of the balloon 104, which extends further from the surface of the catheter in a first radial direction 170 than in a second radial direction 172 opposite the first radial direction. The circumference 173 of the outer surface 166 of the catheter body may be represented as the sum of an exposed fraction 174 that is not covered by the inflated balloon 110 and a covered fraction 176 that projects into the interior volume 140 of the balloon 104. The covered fraction may further include the area occupied by the sealed joint 132 attaching the balloon wall to the outer surface of the catheter. The interior volume 140 of the balloon 104 is bordered by the balloon wall 130 and the covered fraction 176 of the longitudinal outer surface 166 of the catheter body 102.

FIG. 4 shows the balloon 104 in an example of a deflated state 112, folded and stowed against the outer surface 166 of the catheter body 102. FIG. 4 further illustrates an example of a position of the crease 134 along a fold line of the folded balloon 112.

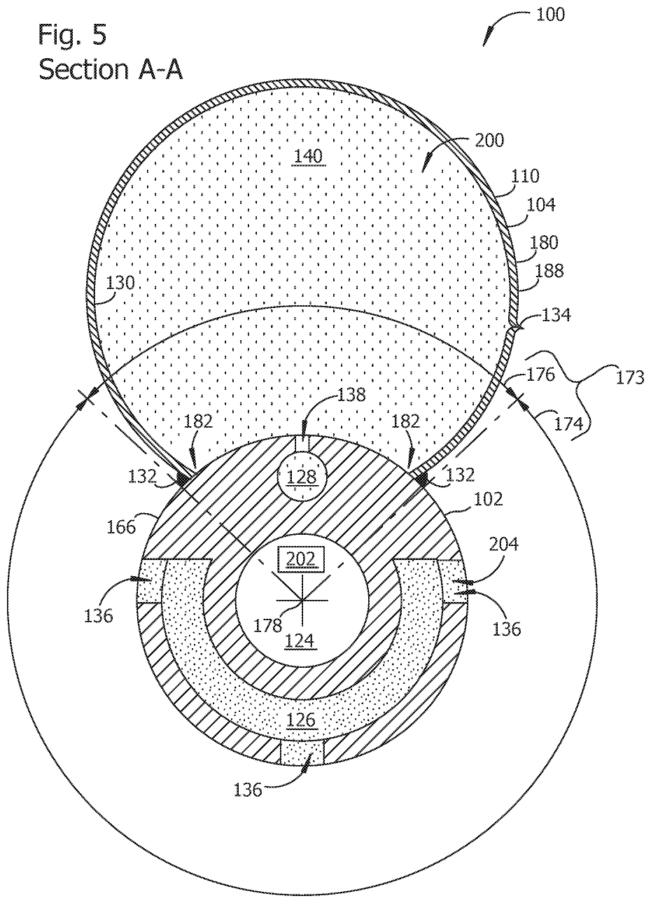

FIG. 5 shows examples of some internal details of the asymmetric balloon catheter 100. The balloon wall 130 of the example of an inflated balloon 110 is sealed against the outer surface 166 of the catheter body 102 by the sealed joint 132. The inflated balloon encloses an interior volume 140 in fluid communication with the first hub 118 through the intervening balloon fill lumen 128 and one or more balloon fill ports 138. Fluid and/or gas may be introduced into the first hub 118 and forced through the balloon fill lumen 128 and balloon fill ports 138 to inflate the balloon. Fluid and/or gas may be withdrawn from the interior volume 140 through the first hub 118 to deflate the balloon. Fluid, for example imaging contrast fluid, may be transported from the third hub 122 to the external fluid ports 136 through the fluid transport lumen 126. Fluid in a body lumen may be drawn into and transported through the external fluid ports 136 and fluid transport lumen 126 by suction applied to the third hub 122, for example by retracting the plunger of a syringe attached to the luer fitting 142 of the hub.

As suggested in the example of FIG. 5, the edges 182 of the balloon wall 130 are attached with a gas-tight and liquid-tight seal to the outer surface 166 of the catheter body 102 on opposite lateral sides of the balloon fill lumen 128. The balloon wall 130 and sealed joint 132 of the inflated balloon 110 preferably do not block the external fluid ports 136, i.e., fluid may flow into and out of the external fluid ports while the balloon is inflated. The external fluid ports 136 are preferably not in fluid communication with the interior volume 140 of the balloon 104 by any lumen formed in the catheter.

The fraction 176 of the circumference 173 of the catheter outer surface 166 covered by the inflated balloon 110, corresponding approximately to the fraction of the circumference projecting into the interior volume 140 of the inflated balloon 110, is about 20% of the total circumference 173 in the example balloon catheter embodiment 100 of FIG. 5. In the example of FIG. 5, about 80% of the total circumference 173 remains exposed outside the illustrated example of a fully inflated balloon 110 and the sealed joint 132. For the example balloon catheter embodiments 100 disclosed herein, the covered fraction is preferably greater than or equal to about 5% of the total circumference of the catheter body to provide sufficient space for the balloon fill ports 138 without the edges 182 of the balloon wall and the sealed joint 132 between the balloon wall 120 and the outer surface of the catheter body blocking the balloon fill ports. The maximum value of the exposed fraction 174 is therefore less than or equal to about 95% of the total circumference for the embodiments 100 disclosed herein.

FIG. 5 further shows an example of a balloon inflation fluid 200 filling the interior volume 140 of the inflated balloon 110, the balloon fill port 138, and the balloon fill lumen 128. Pumping balloon inflation fluid 200 into the balloon inflates the balloon and removing inflation fluid from the balloon deflates the balloon. In FIG. 5, an example of a fluid 204 to be transported to or from a body lumen, for example imaging contrast media, a buffer solution, a body fluid, or a therapeutic compound, is represented by stippling filling the fluid transport lumen 126 and the external fluid ports 136. A schematic representation of an example of a therapeutic or diagnostic instrument 202 is shown in the working lumen 124. Examples of a therapeutic or diagnostic instrument include, but are not limited to, a camera, an optical fiber, a guide wire, a cutting instrument, forceps, and a self-expanding prosthesis. A therapeutic compound, lubricant, or surgical tool may optionally be carried aboard a balloon catheter embodiment by being placed against the outer surface of the catheter body.

Some balloon catheter embodiments 100 have an inflated balloon 110 with a partial cylindrical outer surface 180 blending into a tapered distal end 114 and a tapered proximal end 116 as suggested in the examples of FIG. 1, FIG. 5, and other figures. An inflated balloon 110 may alternately be formed with a partial spherical outer surface 188 as suggested in the example of FIG. 5. A balloon 104 having a spherical outer surface 188 may optionally be formed with tapered distal 114 and/or proximal 116 end caps transitioning the spherical surface to the surface of the catheter.

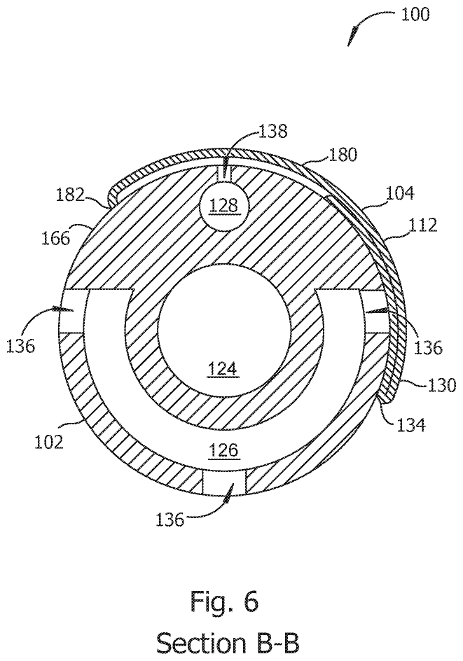

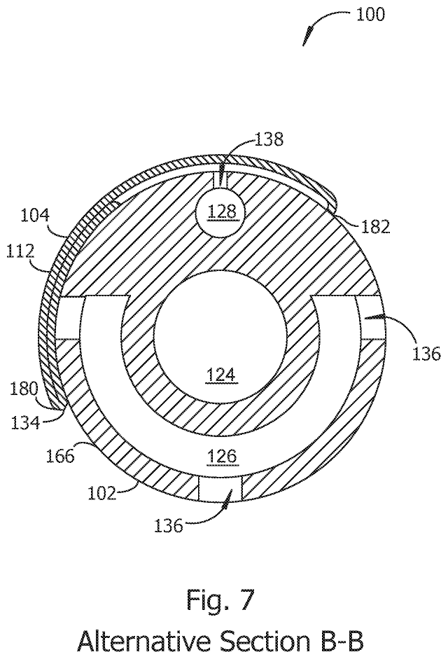

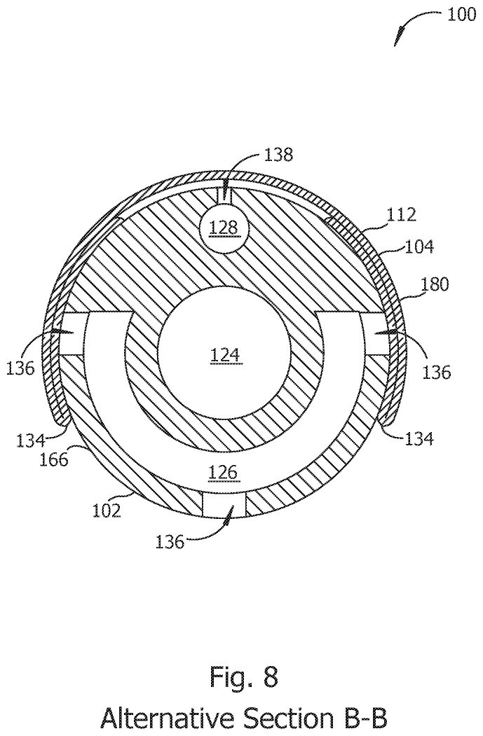

Examples of a balloon 104 in deflated and/or stowed positions 112 are shown in FIGS. 6-8. In the example of FIG. 6, the interior volume of the balloon 104 has been evacuated sufficiently to collapse the balloon onto the outside surface 166 of the catheter body 102. The balloon wall 130 folds along the crease 134 to lie smoothly against the outside of the catheter body with the folded balloon conforming to the shape of the catheter body's surface. The deflated balloon 112 is shown with a single fold line 134 in the examples of FIGS. 5-7 and with two fold lines 134 in the example of FIG. 8.

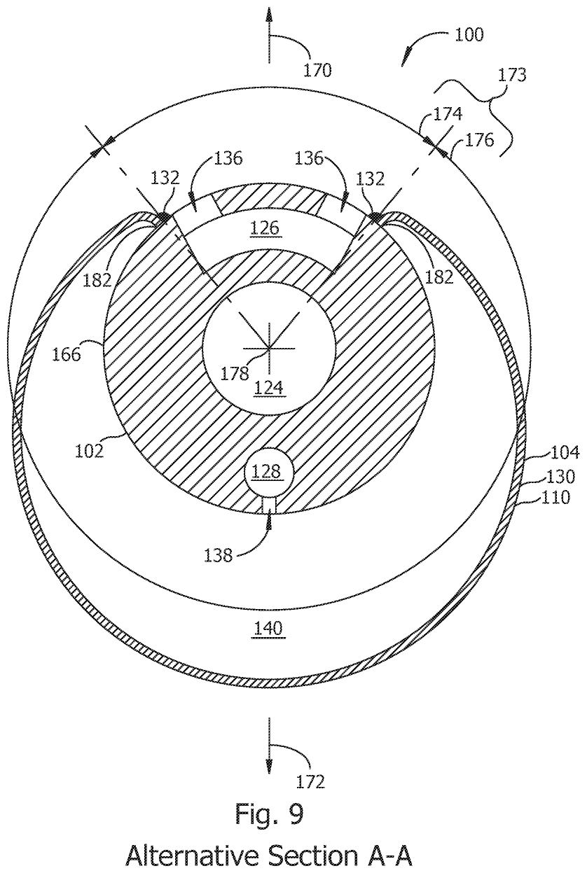

In the examples of an asymmetric balloon catheter 100 in FIGS. 1-8, the exposed fraction 174 of the total circumference 173 of the catheter body 102 may be greater than the fraction 176 covered by the inflated balloon 110. FIG. 9 shows an example of an asymmetric balloon catheter 100 having the exposed fraction 174 substantially smaller than the covered fraction 176. In the example of FIG. 9, the inflated balloon 110 covers about 80 percent of the catheter's total circumference 173, positioning most of the catheter body 102 inside the interior volume 140 of the inflated balloon 110. As with the other example embodiments 100, the inflated balloon 110 and catheter body 102 are not concentric, i.e. do not share a common longitudinal center axis. A covered fraction 176 greater than about 95 percent may interfere with the placement and function of the external fluid ports 136. For some of the example embodiments 100 disclosed herein, the covered fraction 176 is preferably less than about 80% of the total circumference 173 to provide sufficient space for the external fluid ports 136, as suggested in the example of FIG. 9. The minimum size of the exposed fraction 174 is therefore greater than or equal to about 20% of the total circumference 173 for the example of FIG. 9 and greater than or equal to about 5% of the total circumference in general for all balloon catheters in accord with an embodiment 100.

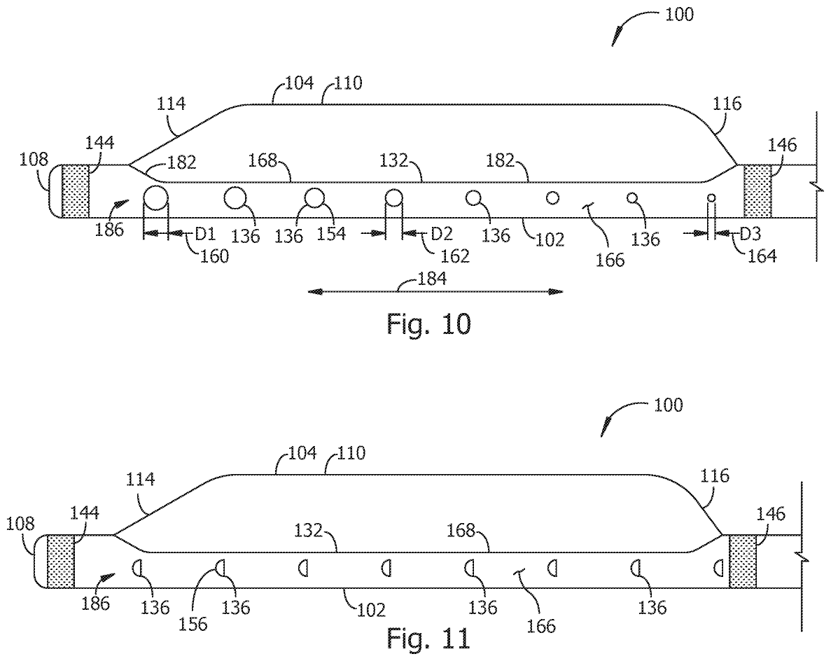

FIGS. 10-14 illustrate examples of alternative shapes, sizes, and separation distances for the external fluid ports 136. The size and shape of external fluid ports 136 and the separation distances between adjacent external fluid ports may be selected to determine the locations where fluids are emitted and recovered from a balloon catheter embodiment, and possibly to establish relative flow rates of fluids through some external fluid ports compared to other ports. For example, the external fluid ports 136 may be formed as cylindrical apertures 154 arranged in a longitudinal row 186 through the outer surface 166 of the catheter body 102. In FIG. 10, the diameter of each example of an external fluid port 136 has been selected to establish approximately equal volumetric flow rates of fluid through each of the external fluid ports 136 in one longitudinal row 186 of external fluid ports 136. For example, some external fluid ports formed as cylindrical apertures 154 with a diameter D1 160, other external fluid ports with a diameter D2 162, possibly with D2<D1, and yet other external fluid ports with a diameter D3 164, possibly with D3<D2. Other balloon catheter embodiments may use a different arrangement of diameters D1, D2, and/or D3.

An example balloon catheter embodiment 100 with different separation distances between adjacent external fluid ports 136 is shown in FIG. 4, where separation distances S1 206, S2 208, and S3 210 may be selected to determine a location of fluid flows into and/or out of the catheter relative to the distal end 108 or the position of the balloon 104. Separation distances S1, S2, and S3 may be equal to one another in some balloon catheter embodiments and different from one another in other balloon catheter embodiments.

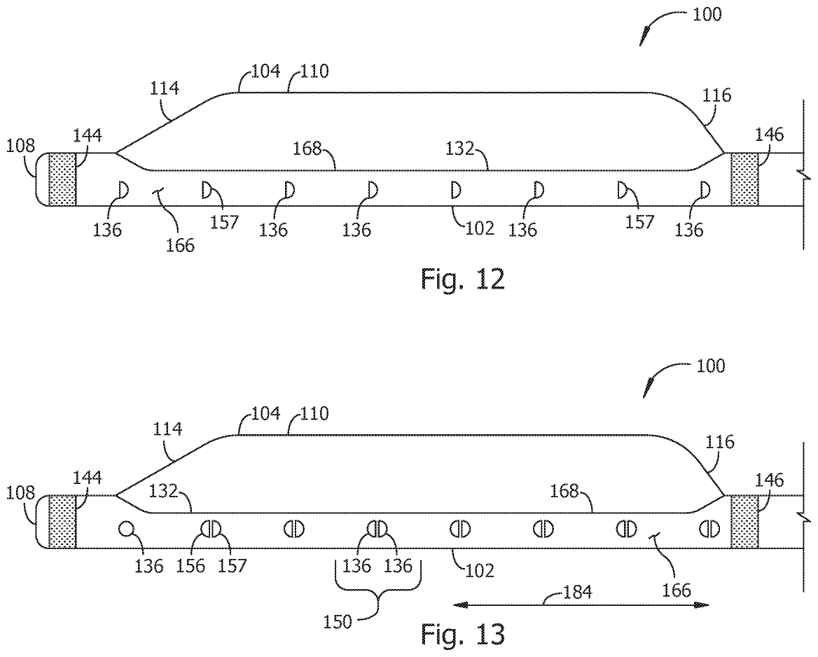

In the examples of FIGS. 11 and 12, the external fluid ports 136 are formed with an arcuate shape 156 or a different arcuate shape 157. In the example of FIG. 13, port groups 150 are spaced in a longitudinal direction 184 along the longitudinal edges 168 of the balloon 104, each port group 150 including at least two external fluid ports 136. The external fluid ports in a port group may have different perimeter shapes from one another as suggested in FIG. 13, where one of the external fluid ports in a port group may have an arcuate perimeter shape 156 and another of the external fluid ports in the same port group may have a different arcuate perimeter shape 157. A balloon catheter embodiment 100 may optionally have more than one size and/or shape of external fluid ports 136.

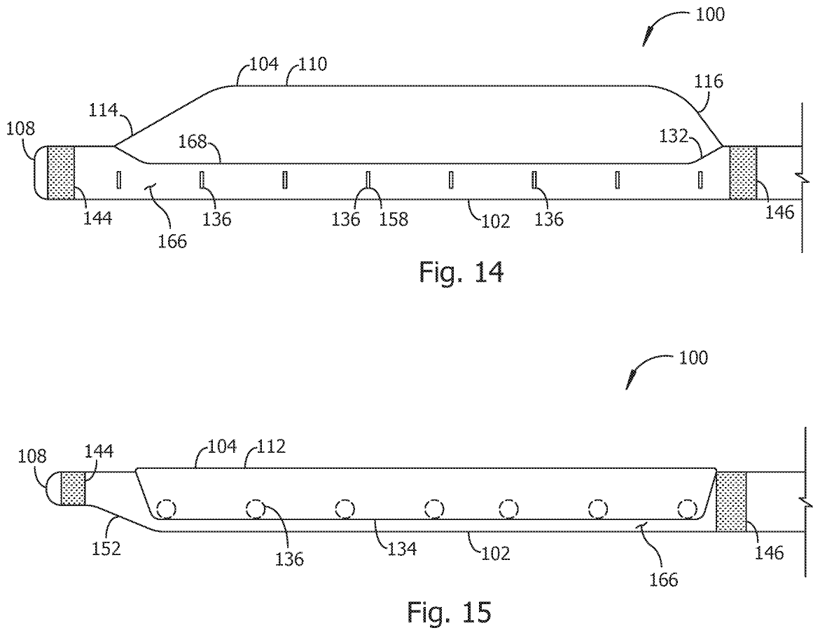

Examples of external fluid ports 136 formed as slots 158 are shown in FIG. 14. The slots 158 may optionally be formed as narrow cuts or slits. The slots 158 may be sufficiently narrow to remain closed unless forced open by the pressure of fluid in the fluid transport lumen 126. After being opened to allow fluid to flow out the external fluid ports 136, reducing pressure in the fluid transport lumen may allow the slots to close, preventing fluid from flowing through the external fluid ports.

The catheter body 102 may optionally be formed with a tapered distal end 152 as suggested in the example of FIG. 15. A tapered distal end 152 may be configured to carry a self-expanding prosthesis or other surgical device on the outer surface of the catheter body. FIG. 15 further shows an example of the balloon 104 in a deflated and/or stowed position 112. In some example balloon catheter embodiments 100, the deflated and folded balloon may cover some of the external fluid ports 136 as suggested in FIG. 4 and FIG. 15.

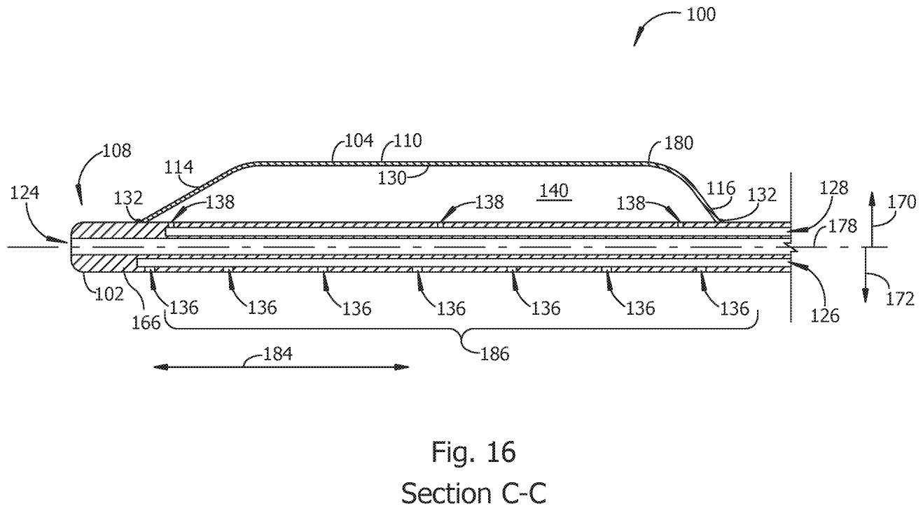

FIG. 16 shows an example asymmetric balloon catheter 100 in a longitudinal cross section C-C. In the illustrated example, the inflated balloon 110 extends outward from the outer surface 166 of the catheter body farther in a first direction 170 than a second direction 172. For some embodiments, the first direction 170 is radially outward from the longitudinal center axis 178 of the catheter body 102 and the second direction 172 is an outward direction opposite the first direction 170. As suggested in the example embodiment 100, the balloon fill lumen 128, the working lumen 124, and the fluid transport lumen 126 are formed in a longitudinal direction 184 through the catheter body 102 and are not in fluid communication with each other within the catheter body. External fluid ports 136 pass through the outer surface 166 of the catheter body to the fluid transport lumen 126. Balloon fill ports 138 pass through the outer surface of the catheter body to the balloon fill lumen 128, establishing fluid communication between the interior volume 140 of the balloon and the balloon fill lumen. The working lumen 124 passes from the second hub at the proximal end 106 through the distal end 108.

Unless expressly stated otherwise herein, ordinary terms have their corresponding ordinary meanings within the respective contexts of their presentations, and ordinary terms of art have their corresponding regular meanings.

What is claimed is:

1. An apparatus, comprising:
a catheter body; and
an inflatable balloon having longitudinal edges sealed to said catheter body, said balloon when inflated configured to extend outward from an outer surface of said catheter body farther in a first direction than a second direction.

2. The apparatus of claim 1, wherein said first direction is radially outward from said outer surface of said catheter body and said second direction is opposite said first direction.

3. The apparatus of claim 1, wherein said catheter body is formed with a balloon fill lumen in fluid communication with an interior volume of said balloon and a working lumen extending from a proximal end of said catheter body to a distal end of said catheter body.

4. The apparatus of claim 3, wherein said catheter body is formed with a balloon fill port in fluid communication with said balloon fill lumen and said interior volume of said balloon.

5. The apparatus of claim 3, wherein said balloon fill lumen is not in fluid communication with said working lumen.

6. The apparatus of claim 3, wherein said catheter body is formed with a fluid transport lumen and a plurality of external fluid ports passing through said outer surface of said catheter body into said fluid transport lumen.

7. The apparatus of claim 6, wherein said catheter body is formed with said plurality of external fluid ports formed in said catheter body adjacent a first longitudinal edge of said balloon.

8. The apparatus of claim 6, wherein said catheter body is formed with said plurality of external fluid ports positioned in said catheter body opposite said balloon.

9. The apparatus of claim 6, wherein said fluid transport lumen is not in fluid communication with said working lumen.

10. The apparatus of claim 6, further comprising a luer assembly attached to said catheter body, said luer assembly comprising:
a first hub in fluid communication with said balloon fill lumen;
a second hub in fluid communication with said working lumen; and
a third hub in fluid communication with said fluid transport lumen.

11. The apparatus of claim 6, wherein said balloon when inflated does not cover said external fluid ports.

12. The apparatus of claim 3, wherein said balloon is formed with a tapered distal end and a tapered proximal end.

13. The apparatus of claim 1, further comprising a radiopaque marker positioned on said outer surface of said catheter body between said balloon and said distal end of said catheter body.

14. The apparatus of claim 1, further comprising a radiopaque marker positioned on said outer surface of said catheter body adjacent a proximal end of said balloon.

15. The apparatus of claim 1, wherein said balloon when inflated covers less than eighty percent of a circumference of said catheter body.

16. The apparatus of claim 15, wherein said balloon when inflated covers at least twenty percent of said circumference of said catheter body.

17. The apparatus of claim 16, wherein said catheter body is formed with a balloon fill lumen in fluid communication with an interior volume of said balloon, a fluid transport lumen, and a plurality of external fluid ports passing through said outer surface along said at least twenty percent of said circumference into said fluid transport lumen.

18. The apparatus of claim 1, wherein said catheter body is formed with a first external fluid port having a first diameter and a second external fluid port having a second diameter less than said first diameter.

19. The apparatus of claim 1, wherein said catheter body is formed with an external fluid port shaped as a narrow slit, said slit configured to remain closed unless forced open by sufficient pressure of a fluid in said catheter.

20. The apparatus of claim 1, wherein said catheter is formed with a balloon fill lumen, a fluid transport lumen, a working lumen, and a plurality of external fluid ports passing through a portion of an outer surface of said catheter not covered by said balloon when inflated, said catheter configured for simultaneous transport of a first fluid through said balloon fill lumen, a medical instrument through said working lumen, and a second fluid through said fluid transport lumen.
